(19) United States
(12) Patent Application Publication
(10) Pub. No.: US 2004/0115672 A1
(43) Pub. Date: Jun. 17, 2004

(54) Use of electrical fields to control interactions between proteins and nucleic acid constructions immobilized on solid supports

(76) Inventors: Rastilav Levicky, New York, NY (US); Patrick Johnson, New York, NY (US)

Correspondence Address: BAKER & BOTTS, 30 ROCKEFELLER PLAZA, NEW YORK, NY, 10112

(21) Appl. No.: 10/465,940

(22) PCT Filed: Feb. 1, 2002
(86) PCT No.: PCT/US02/03943

(51) Int. Cl. C12Q 1/68

(52) U.S. Cl. 435/6

(57) ABSTRACT

The present invention relates to devices and methods for controlling molecular reactions involving nucleic acids, such as transcription, by controlling the conformation of nucleic acids bound to a solid support. This is accomplished by applying an electrical bias to a conductor surface which gives rise to a nonspecific attraction or repulsion between the bound nucleic acids and the conductor surface resulting in increased or reduced adsorption of the nucleic acid on the conductor surface. When the nucleic acids are adsorbed onto the conductor surface, their backbone is less accessible to the binding of factors, such as transcription factors, conversely, when the nucleic acids are released from the conductor surface (preferably leaving some interaction with the support) their backbone becomes more accessible to the binding of factors. The ability to control the conformation of nucleic acids on the conductor surface and the resultant control of the binding of factors to the nucleic acids, allows for a system, such as a gene transcription system, which may be turned off and on. Local control of surface charge may be achieved by using electronically addressable pads arranged in an array or microarray format.

### SPECIFICATION

This application claims priority to U.S. Provisional Application No. 60/266,118, filed on Feb. 1, 2001 which is incorporated herein in its entirety by reference.

### BACKGROUND OF THE INVENTION

This invention relates to devices and methods for controlling molecular reactions involving nucleic acids by controlling the conformational state of the nucleic acids when bound to a solid support. The conformational state of the nucleic acids bound to the solid support is controlled by applying an electrical bias to the solid support.

Recent advances in high-throughput biomolecular technologies include oligonucleotide and complementary DNA (cDNA) microarrays for gene expression and variation studies (Schena M et al. 1998, Trends Biotechnol. 16(7):301-306; Southern EM, 1996, Trends Genet. 12(3):110-115; Lockhart DJ et al., 2000, Nature 405(6788):827-836), protein chips for studying protein interactions with drugs, substrates, and other proteins (MacBeath G et al., 2000, Science 289(5485):1760-1763), and self-contained micro-analytical devices for processing and analyzing a variety of biochemical and chemical matter (Mastrangelo CH et al., 1998, Proc. IEEE 86:1769-1787; Sanders GHW et al., 2000, Trends Anal. Chem. 19:364-378).

Microanalytical methods for the investigation of biomolecules (e.g., nucleic acids, proteins) are rapidly evolving from proof-of-concept experiments into robust measurement systems for high-throughput analysis. To this end, a great deal of effort has been made to develop multiplex DNA sensors, so-called DNA chips (Cheung V et al., 1999, Nat. Genet.(suppl.)21:15-19; Duggan D et al., 1999, Nat. Genet.(suppl.)21:10-14; Lipshutz RS et al., 1999, Nat. Genet.(suppl.)21:20-24). At the core of DNA chip technology are arrays of single-stranded DNA (ssDNA) chains, or probes, that are tethered to a substrate for capture of complementary analyte nucleic acids, or targets. For DNA chip technology to continue to emerge as an alternative to conventional DNA diagnostic methods, questions about the conformation and activity of DNA attached to surfaces must be addressed (Kelley SO et al., 1997, Bioconjugate Chem. 8:31-37; Chan V et al., 1997, Langmuir 13:320-329; Williams JC et al., 1994, Nucleic Acids Res. 22:1365-1367).

The two predominant methods of producing surface-immobilized probes are direct, on-chip synthesis of nucleic acids (Pease AC et al., 1994, Proc. Natl. Acad. Sci. USA 91:5022-5026; Southern EM et al., 1994, Nucleic Acids Res. 22:1368-1373) and attachment of presynthesized oligonucleotides that are chemically modified to effect surface immobilization (O'Donnell MJ et al., 1997, Anal. Chem. 69:2438-2443; Gingeras TR et al., 1987, Nucleic Acids Res. 15:5373-5390). Although the former method presents an elegant approach to chip fabrication, it requires resources and expertise that can limit facile implementation. In addition, it is difficult to non-destructively characterize probes (e.g. probe length) produced with an on-chip synthesis approach (Southern E et al., 1999, Nature Genetics 21(1 Suppl.):5-9). In an alternate approach, the use of presynthesized probes modified with an appropriate surface-linking group is also common (Chrissey et al., 1996; Guo Z et al., 1994, Nucleic Acids Res. 22:5456-5465; Pirrung MC et al., 2000, Langmuir 16:2185-2191). Irrespective of how DNA chips are fabricated, a greater understanding of the factors influencing the structure of immobilized DNA layers is needed to design surfaces exhibiting greater biological activity and selectivity.

One suitable model system for fundamental studies consists of thiol-containing nucleic acid probes that are immobilized through self-assembly to gold surfaces, as recently employed by several investigators (Bamdad C, 1997, Biophys. J. 75:1989-1996; Bamdad C, 1997, Biophys. J. 75:1997-2003; Levicky R et al., 1998, J. Am. Chem. Soc. 120:9787-9792; Herne TM et al., 1997, J. Am. Chem. Soc. 119:8916-8920; Peterlinz KA et al., 1997, J. Am. Chem. Soc. 119:3401-3402). For example, scanning tunneling microscopy images of oligonucleotides on gold have reported that single-stranded DNA (ssDNA) appeared as “blobs,” whereas double-stranded DNA (dsDNA) was rod-like (Rekesh DY et al., 1996, Biophys. J. 71:1079-1086). Typically, when gold surfaces are used the ssDNA probes are attached through a sulfur-gold linkage.

Recent neutron reflectivity studies indicated that on bare gold, ssDNA oligonucleotides form a compact layer—a picture that is consistent with the presence of multiple contacts between each strand and the substrate (Levicky R et al., 1998, J. Am. Chem. Soc. 120:9787-9792). DNA nucleotides can presumably adsorb to gold via multiple amine moieties, as amines are known to chemisorb weakly to gold surfaces (Xu CJ et al., 1993, Anal. Chem. 65:2102-2107; Leff DV et al., 1996, Langmuir 12:4723-4730). Such adsorption at multiple sites can interfere with subsequent interactions, such as hybridization of the immobilized strands to other ssDNAs. As a remedy, the accessibility of immobilized probes to complementary target sequences can be enhanced by treating the surface with a small-molecule blocking agent, 6-mercapto-1-hexanol (MCH). The thiol group of MCH rapidly displaces the weaker adsorptive contacts between DNA nucleotides and the substrate, leaving the probes tethered primarily through the thiol end groups. After MCH treatment, the initially compact ssDNA swells and extends further into solution (Levicky R et al., 1998, J. Am. Chem. Soc. 120:9787-9792). The less constrained tethering geometry renders the probes highly accessible to target, with nearly complete hybridization efficiencies observed (Steele AB et al., 1998, Anal. Chem. 70:4670-4677).

U.S. Pat. No. 5,849,486 to Heller et al., entitled “Methods for Hybridization Analysis Utilizing Electrically Controlled Hybridization” (hereinafter “the '486 patent”), discloses a device that is able to control and actively carry out a variety of biomolecular assays and reactions. Reactants may be directed to specific locations on the device by free field electrophoresis, thus concentrating the reactant at the micro-location. Unbound reactants are removed by reversing the polarity of a micro-location which results in improved specificity of the reactions at micro-locations. The device allows for the control of reactions by allowing for the timed release of reactants. While the '486 patent relates to controlling biomolecular reactions, it does not relate to a method for improving a biomolecular reaction by altering the conformational state of nucleic acid molecules bound to the solid support.

U.S. Pat. No. 6,120,985 to Laugharn, Jr. et at., entitled “Pressure-enhanced Extraction and Purification” (hereinafter “the '985 patent”), discloses modulating the binding of a nucleic acid to a solid support by modifying the pressure level such that the modification disrupts the binding of the nucleic acid to the solid support. This is an all or none reaction. The DNA is bound to the solid support and the application of pressure causes the release of the DNA.

Similarly, U.S. Pat. No. 6,127,534 to Hess et al., entitled “Pressure-modulated Ion Activity” (hereinafter “the '534 patent”), discloses controlling chemical reactions, including catalytic reactions and association/dissociation reactions by modulating the ionic activity of the solution which, in turn, changes the rate of the reaction. The ionic activity is modulated by changing the pressure. The '534 patent teaches that modification of pressure may dissociate a sample from a solid support. Dissociation is considered useful to isolate the sample from the support or to regenerate the support.

Nilsson J et al. discloses controlling a nucleic acid synthesis reaction (the polymerase chain reaction) using an immobilized DNA polymerase on a solid support. See Nilsson J et al., 2000, Biotechniques 22(4):744-751. Heat-mediated elution of the DNA polymerase from the solid support allows for a temperature-induced activation of the DNA polymerase which is inactive when bound to the solid support. The temperature induced activation results in a controlled polymerase chain reaction which requires that the DNA polymerase bind to a primed nucleic acid template. Once the polymerase is activated, no further means of control is available.

Kelley et al. attached thiol-terminated, double-stranded DNA 15 mers to gold electrodes via the thiol end group. See Kelley SO et al., 1998, Langmuir 14:6781-6784. Using atomic force microscopy, it was observed that the duplexes could be driven to stand straight up or to lie flat on an electrode support surface depending on the electrical bias applied to the electrode support. By varying the surface potential by 100 mV, a complete transition from the standing up to the lying flat orientation of the immobilized DNA could be reversibly triggered. However, Kelley et al. does not relate to controlling the interaction of biomolecules with DNA on the basis of the interaction of the DNA with a surface.

Microfabricated heater pads have been demonstrated to thermally control local enzyme activity and therefore expression of surface-bound complementary DNA (cDNA), demonstrating one approach to purposefully directed gene expression (Shivashankar GV et al., 2000, Appl. Phys. Lett. 76:3638-3640). The present invention further advances the capability for in vitro enzymatic processing of immobilized nucleic acids by developing electronic control (by modulating the charge on the surface to which nucleic acid molecules are attached) to improve such active arrays by increasing the density of array sites, enhancing the ease of operation of such devices and arrays, and enabling facile integration into self-contained analytical devices.

### SUMMARY OF THE INVENTION

The present invention relates to methods and devices for controlling the degree of contact between an electrode surface-immobilized nucleic acid and a surface to which it is attached through electrical fields generated on the electrode surface and the charge on such surface. The present invention further relates to methods and devices for on-command control over processing surface-immobilized nucleic acids by proteins or other molecules. In accordance with the invention, control is realized, in part, through application of an electrical bias to the surface. A more positively charged surface will nonspecifically attract the negatively charged phosphate backbone of the nucleic acid consequently decreasing its availability to bind polymerases or other molecules. On the other hand, a more negatively charged surface will repel the negatively charged phosphate backbone of the nucleic acid leading to increased exposure of the nucleic acid to solvent and any molecules suspended or dissolved therein.

### BRIEF DESCRIPTION OF THE DRAWINGS

The present invention may be better understood with reference to the attached drawings in which:

FIG. 1 shows a schematic showing adsorption of an immobilized complementary DNA (cDNA) chain, induced by positively biasing the electrode (left to right), thus obstructing RNA polymerase, a processing enzyme, from scanning the cDNA to locate the promoter and initiate transcription;

FIG. 2 shows a schematic showing a prototype array of electrodes, each bearing cDNA coding for a unique protein; and

FIG. 3 shows a schematic showing (A) a PCR primer is shown next to its binding site on a single-stranded DNA (ssDNA) molecule, (B) the ssDNA attached to a surface at either its 5′ or 3′ end, (C) a more negative surface charge that exposes the binding site only on the 5′ anchored DNA, and (D) a still more negative surface charge that exposes the binding site for both the 5′ and 3′ anchored DNA molecules.

### DETAILED DESCRIPTION OF THE INVENTION

The present invention relates to devices and methods for controlling reactions by controlling the conformation of nucleic acids bound to a solid support by adjusting the surface charge. Reactions that may be susceptible to such control include, for example, transcription, translation, ligation, duplication, and digestion. The degree of interaction between the surface-immobilized nucleic acid and molecules in solution may be modulated by controlling the conformation of the nucleic acid. A more negative surface charge will repel the negatively charged phosphate backbone of the surface-immobilized nucleic acid such that the surface-immobilized nucleic acid is more exposed and accessible to solvent molecules and molecules in solution. Correspondingly, a more positive surface charge will result in greater adsorption of the surface-immobilized nucleic acid to the surface, thereby restricting contact with solvent and molecules in solution.

In some embodiments of the invention, the association of the nucleic acid with the surface is further modulated by modifying the electrochemical potential of the nucleic acid by any means known in the art. For example, the degree of protonation of the phosphate backbone or nucleic acid bases may be regulated by adjusting the pH to be higher or lower than the corresponding pI. In addition, covalent modification of some or all of the phosphate hydroxyl groups, or the nucleic acid bases, may modify the electrochemical potential of the nucleic acid. Contacting the nucleic acid with a detergent may also modify the electrochemical potential of the nucleic acid. Furthermore, oligonucleotides or polynucleotides having a modified phosphate or non-phosphate (e.g. sulfur) backbone may have an altered electrochemical potential.

According to the invention, nucleic acids that may be immobilized on a surface may be single or double stranded. They may comprise RNA and/or DNA, and may originate from naturally occurring or artificially prepared nucleic acid molecules. They may comprise coding and/or noncoding regions. The coding region may be operably linked to expression control sequences. A plurality of noncoding regions, coding regions and expression control sequences may be combined on one nucleic acid molecule.

According to the invention, nucleic acids are polynucleotides, typically between 10 bases and 10 kilobases in length. In one embodiment, the nucleic acid is from about 100 bases to about 10 kilobases. Shorter and longer polynucleotides are also within the scope of the invention. However, in situations in which the nucleic acids are covalently immobilized to the solid support (e.g. by one end) complications may arise as length increases due to difficulty with strict maintenance of the attachment geometry. Very long nucleic acids carry a large quantity of native functional groups that may provide increased competition, through side reactions, to the desired immobilization chemistry. An additional complication that may arise as a function of length is undesired intra-strand and inter-strand hybridization or formation of other undesired, non-native structures. Such structures may interfere with the association of transcription factors, polymerases, or other molecules. These complications may be remedied or ameliorated by adjusting salt conditions, through addition of denaturing or chaotropic agents such as tetramethyl ammonium chloride, by changing the temperature, or by taking other steps that can influence interactions between the molecules participating in the reaction and the nucleic acids.

In some embodiments of the invention, at least one nucleic acid is immobilized on an electrode surface. The nucleic acid may be attached to the electrode surface by a plurality of chemical bonds. Preferably, the nucleic acid is immobilized on an electrode surface by at least one covalent chemical bond.

A central principle of the invention is that enzymatic manipulation of surface-bound nucleic acids may be controlled by adjusting the charge on the electrode surface to which the nucleic acids are immobilized. The requisite control over nucleic acid conformation may be realized at realistic surface charge densities. For a charged polymer such as a nucleic acid, electrostatically-induced adsorption to a charged surface is expected if (Muthukumar M, 1987, Macromolecules 86:7230-7235; Kong CY, 1998, J. Chem. Phys. 109:1522-1527):

s≧0.038(εbb_{K}K³kT)/|q|

where

s is surface charge density,

ε is the bulk solvent electric permittivity,

b is the length of a nucleic acid statistical segment,

b_{K} is a statistical segment length adjusted for intersegmental interactions,

K is the inverse of the Debye electrostatic screening length,

k is the Boltzmann constant,

T is temperature, and

q is charge per nucleic acid statistical segment.

At physiological salt concentrations, b_{K}≈b. It is noted that a critical surface charge density is predicted before adsorption occurs.

At room temperature and 0.1 M ionic strength, allowing for possible effects of counterion condensation on the effective charge of a nucleic acid chain (Bloomfield VA et al., 2000, Nucleic Acids—Structures, Properties, and Functions. University Science Books (Sausalito) p.491), adsorption is predicted for s>˜0.05 C/m². From the Grahame equation (Israelachvili, 1992, Intermolecular and Surface Forces. Academic Press (San Diego) p.234), this charge density corresponds to a 57 mV surface potential (relative to the potential of zero charge of the surface). Thus, relatively modest surface potentials should be sufficient to induce adsorption even if the effective charge of the polymer is lowered by counterion condensation; indeed, similar magnitudes of surface potential were reported in a recent study using electric fields to orient 15 mer oligonucleotides tethered to gold electrodes (Kelley SO et al., 1998, Langmuir 14:6781-6784).

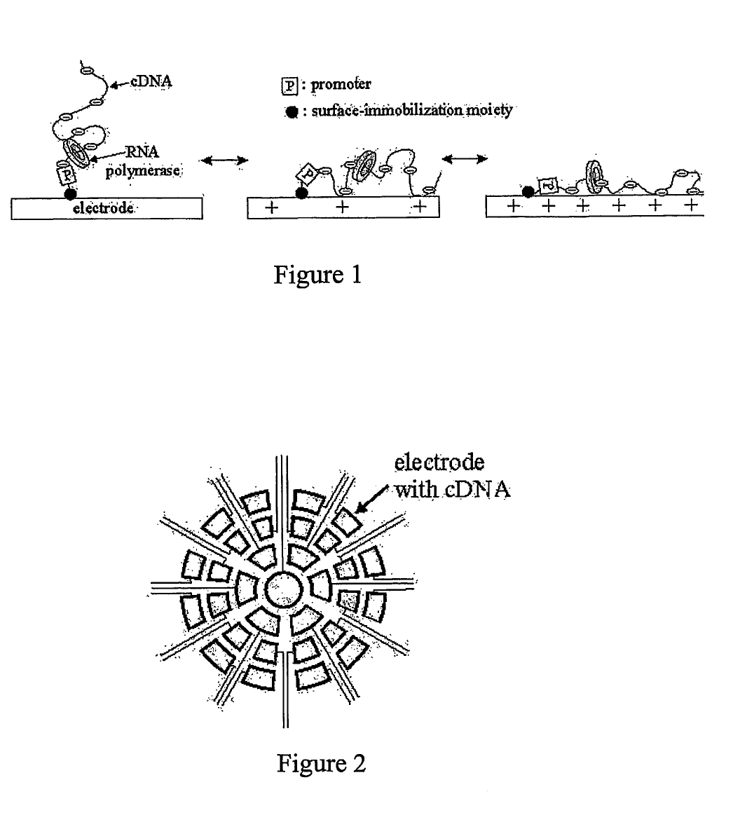

In accordance with the present invention, an electrode surface is provided comprising at least one surface-immobilized nucleic acid wherein said surface may be charged and wherein said nucleic acid may be bound to said surface through electrostatic attraction. In one embodiment, the surface is arranged in electronically addressable pads (FIGS. 1 and 2 and Example 2 below).

Devices of the invention comprise a surface made of any material that is capable of accepting a positive or negative charge. In some embodiments, the electrode surface comprises metal such as platinum, gold, silver, copper, aluminum or tin. The chemical stability of platinum and/or gold may render them particularly suited for metal electrodes. In some embodiments, the surface is an indium tin oxide or highly doped, conductive silicon electrode. A potentiostat, rheostat or other variable resistance device connected in series between a voltage source and the electrode may be used to regulate the potential applied to the electrode and the charge on the electrode surface. Devices of the invention may further comprise a coating material on the electrode surface (e.g. various organothiols; also synthetic polymers such as polyacrylic acid, polyacrylamide, polyethyleneoxide) that is capable of reducing the association between the electrode surface and at least one molecule or class of molecules in solution. The electrode may be fabricated by any means known in the art including, for example, thermal evaporation or sputter deposition onto a solid (e.g. glass) support.

The devices of the invention may be configured, for example, such that nucleic acids may be individually deposited on electrode pads. These pads may be capable of independent or coordinated control with respect to immobilization of nucleic acids and/or electrical charging. Devices of the invention may be combined with microfluidic devices, such as those described by Mastrangelo et al. and Sanders et al., to develop novel micro-total-analysis systems. Devices of the invention may also be combined with microfabricated analytic systems such that the combination is capable of applications including biomolecular sensing, catalysis, drug design, and analysis of gene and protein function.

The invention is not limited by the format of nucleic acid immobilization. Homogeneous or heterogeneous mixtures of nucleic acids may be immobilized on an electrode surface. Nucleic acids may be immobilized in array format, array-like format, or any other format. For example, microarrays may be prepared comprising more than 250,000 different oligonucleotide probes or 10,000 cDNAs per square centimeter (Lipshutz RS et al., 1999, Nat. Genet.(suppl.)21:20-24; Bowtell DD, 1999, Nat Genet. 21(1 Suppl):25-32).

Electrode patterns can be readily miniaturized and multiplexed (i.e. in an array format) using existing microfabrication technology. This scalability is essential to compact surface attachment and simultaneous analysis and/or processing of large classes of nucleic acids such as genomes, combinations of genomes, gene categories (e.g. antibody genes).

Similarly, independent control of nucleic acid processing (e.g. transcription) is an essential feature for large scale gene expression analysis. Significantly enhanced density of array sites (˜two orders of magnitude) compared to thermally-based systems may be possible since temperature “hot spots” are more difficult to localize due to diffusion of heat.

Finally, integration with microfluidic devices may be readily realizable in a “lab-on-a-chip” format.

The present invention further provides a method for controlling the adsorption of at least one nucleic acid to a surface comprising applying an electrical bias to a surface wherein said nucleic acid is anchored to the surface and wherein the adsorption of said nucleic acid to the electrode surface is controlled by the resulting surface charge thereon. According to the invention, anchor refers to a covalent or non-covalent bond between a surface and a nucleic acid which remains intact (i.e. keeps the nucleic acid bound to the surface) under all electrostatic conditions of a particular application. In one embodiment of the invention, contact between an anchored (surface-immobilized) nucleic acid and a surface may be controlled by adjusting the surface charge on the surface wherein a more negative charge decreases the adsorption of the nucleic acid with the surface and a more positive charge increases the adsorption of the nucleic acid with the surface (FIG. 1). Control of the adsorption of the nucleic acid to the surface allows for the control of reactions in which the nucleic acid is involved.

Accordingly, the method may further comprise contacting the surface-immobilized nucleic acid with a solution. The solution may comprise water, a buffer, a nucleic acid, a protein, an enzyme, a carbohydrate, a lipid, an salt, a salt ion, an organic chemical, a drug, a detergent, or combinations thereof. For example, the surface-immobilized nucleic acid may be contacted with a solution containing the requisite RNA polymerase, nucleotide triphosphates, and other ingredients to support transcription. In some embodiments of the invention, the immobilized nucleic acid may be exposed to whole cell extract or a partially purified fraction thereof. In other embodiments, the immobilized nucleic acid may be exposed to whole cells.

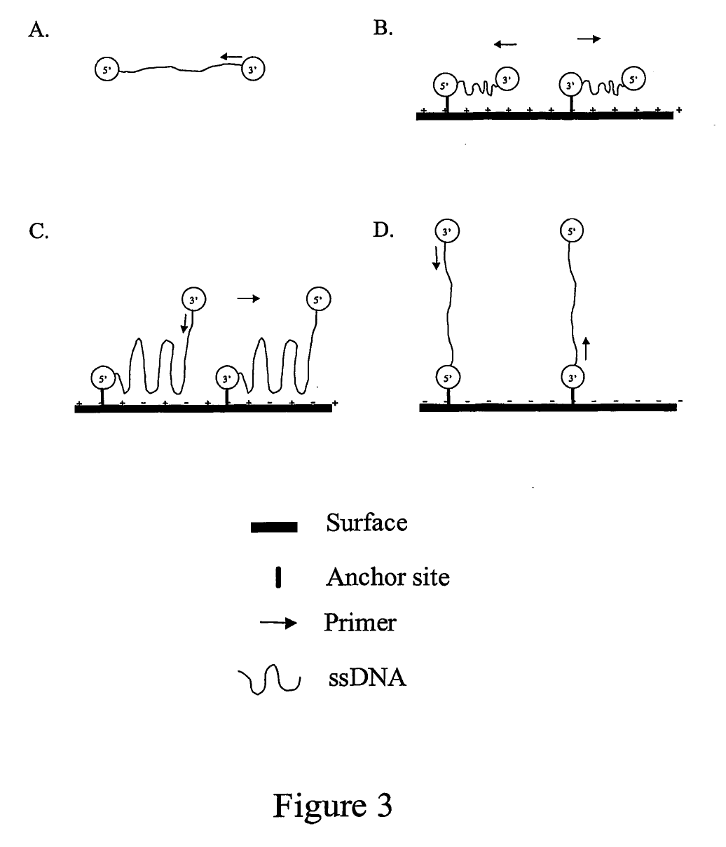

The methods of the invention may be used to modulate a polymerase chain reaction (PCR) to achieve amplification of specific nucleic acids. For example, surface charge may be used to control access of primers and/or polymerase to the template, particularly in the first round of amplification. The location of the attachment site may also affect amplification. FIG. 3 shows that a single-stranded DNA molecule may be attached at either its 5′ or 3′ end according to the invention. This point of attachment (anchor site) may be a covalent or non-covalent linkage. When the surface charge is sufficiently positive neither primer has access to its binding site (FIG. 3B). A more negative surface charge may expose a distal but not a proximal primer binding site relative to the anchor site (FIG. 3C). A still more negative surface charge exposes both proximally and distally situated binding sites (FIG. 3D). Similarly, the binding of polymerase may also be controlled. Such regulation of primer and polymerase binding can regulate DNA amplification and may substantially reduce PCR artifacts such as those due to mispriming. For example, by orienting a template nucleic acid molecule such that a degenerate or low stringency primer binding site is located at the distal end, mispriming at internal or proximal regions of the template may be reduced.

The invention allows for, inter alia, in vitro processing of nucleic acids (e.g. transcription, translation, modification, ligation, and recombination); artificial control over expression of immobilized nucleic acids; study of biochemical regulatory processes and pathways; screening, discovery, and refinement of protein function; and sensing.

By controlling the binding of enzymes, such as polymerases, ligases, restriction enzymes, and nucleases, to surface-immobilized nucleic acids, active control over processing of the nucleic acids may be realized. This capability may be valuable for discovery, manipulation, and interpretation of genetic information. For example, by modulating polymerase action on immobilized DNA, the invention may complement existing methods for detecting sequence polymorphism by single-nucleotide extension (Nikiforov TT et al., 1994, Nucleic Acids Res. 22:4167-4175; Pastinen T et al., 1997, Genome Res. 7:606-614). In such an application, a target nucleic acid containing a polymorphic site may be first hybridized to surface-tethered probe DNA, the sequence of which extends up to, but does not include, the polymorphic site. Polymerase then extends the probe by a single (dideoxy) nucleotide to detect the corresponding polymorphic base in the target. The present invention may enable such measurements to be carried out in duplicate without interrupting contact with the sample solution, therefore maximizing reproducibility and control over experimental parameters. This could be achieved by having the same type of probe on two or more electrode pads, but only using a single pad at a time. Activity of other pads in any given trial would be shut down by blocking the interaction between polymerase and the immobilized nucleic acids through surface-charge driven adsorption of the nucleic acid. The ability to rapidly repeat identical or related measurements without interrupting contact with a sample solution may significantly improve flexibility of experimental design and accuracy of sequence determination and discrimination. Such experimental flexibility may be of especial benefit whenever multiple trial runs are warranted because of the difficulty of the experiment or ramifications of inaccurately determined information (e.g. as in patient genotyping).

A particularly powerful application of the invention involves use of surface potential to control access of RNA polymerases to immobilized genes (FIG. 1) such that transcription of the gene is tuned. Since transcription is an essential step in gene expression, in vitro control over gene expression can be achieved. Gene expression is the biochemical process by which genetic information in genes is transcribed and translated into the amino acid sequence of the corresponding protein. Implemented at a genome wide scale, control over the gene expression patterns of a set of genes corresponding to an entire organism may be realizable.

Such on-command, electronic control of gene expression according to the invention refers to the ability to influence (e.g. initiate, stop, attenuate, or amplify) transcription or transcription and translation of a nucleic acid sequence almost immediately (e.g. within minutes or less) following application of an electrical bias to an eletrode surface. This level of control may enable in vitro simulation of biochemical processes. For example, by creating a pad array of genes (e.g. cDNA molecules, gene constructs) that comprise a gene regulatory circuit or network, in which some of the protein products of the genes influence the expression of other genes in the network pathways, the unique function of a gene involved in the network may be investigated through examining the response of the network reactions to modulations of the expression of said gene. The expression of said gene would be modulated by adjustment of its transcription via potential bias of the electrode to which the gene is immobilized. In addition to deducing gene function in a regulatory network, the role of transcription factors, repressors, and other biological or synthetic molecules (e.g. drugs) involved in controlling gene expression may be deduced, discovered, or improved using such in vitro, artificial gene expression regulatory networks as experimental platforms. For example, it may allow for the identification of proteins involved in gene expression by allowing the isolation of such proteins during different stages of gene transcription and translation.

The ability to control the accessibility of the genes may further allow analysis of the kinetics of gene expression and cascades of gene expression, and reveal quantitative and qualitative information about the kinetics and thermodynamics of the interaction and reaction of specific processing enzymes (e.g. polymerases, restriction enzymes, ligases, nucleases) with nucleic acids.

The methods and devices of the invention may further be useful for applications that benefit from the ability to express functional protein fragments. For example, arrays of natural or artificial gene constructs bearing one or more coding regions such as exons and introns on a single nucleic acid molecule, as well as associated promoter and regulatory sequences, may be designed to express families of antibody fragments for immunological investigations. The methods and devices of the invention may permit microproduction of a vast number of antibody fragments which could then be individually tested for binding affinity to a target molecule.

Similarly, the invention may be useful as a system for synthesizing active enzyme domains to be used in catalyst discovery in native and non-native reactions. For example, first, members of the cytochrome P450 gene superfamily may be attached to a surface. Polypeptides may be produced according to the methods of the present invention and individually tested to determine whether they possess a particular activity. For example, a substrate may be provided, such as a native substrate (e.g. a naturally occurring hormone or lipid) or non-native substrate (e.g. a drug or a toxin) and a binding reaction may be performed to determine whether the polypeptide binds the substrate. In addition to or instead of binding assays, the polypeptides may be tested to determine whether they possess catalytic activity towards the substrate provided. This may lead to the discovery of more effective drugs and a better understanding of the P450 superfamily.

Methods and devices of the invention may be useful in molecular sensing applications. The on-command ability to produce proteins or peptides may be useful in applications where peptide or protein stability or availability limits the durability or efficacy of a sensor.

Key technological advantages of the invention include responsiveness, scalability, independent controls, and compatibility. Since surface potential can be adjusted virtually instantaneously, rapid adjustment of enzymatic processes may be possible. For example, the ability to adjust gene expression allows for implementation of computer-mediated feedback controls on the basis of gene product accumulation or some secondary event. Thus a computer-based program could be used to take over part or all of the feedback mechanisms present in a biochemical pathway. Such capability may be used in studies aimed at understanding biological reaction networks (including gene regulatory networks described above), or in probing the effect of a chemical agent (such as a drug or hormone) on a biochemical regulatory network. The knowledge gained through such experiments may lead to improved fundamental understanding of living systems, including “decision-making” processes in which a living system uses chemical input to determine a response or course of action as reflected in an adjustment of the pattern of gene expression. A related example is sensing, in which the methods and devices of the invention may lead to improved “smart” sensing in which an initiatory external signal (e.g. presence of an analyte) is used by a computer program to determine and initiate a secondary response (e.g. one designed to further screen and identify the analyte detected). For example, such a secondary response could be mounted by triggering the expression of peptides or RNA fragments whose interaction with the analyte can further identify the analyte's chemical nature. A sensing device may be constructed in which classes of such RNA or peptide molecules are encoded by DNA chains immobilized on arrays of electrode pads, with the expression of each RNA or peptide triggered when needed through the methods of this invention.

### EXAMPLES

The present invention is illustrated by, but not limited to, the following examples. Other examples and embodiments will be apparent to those skilled in the art without departing from the spirit and scope of the invention, the scope being defined by the appended claims.

### Example 1

Devices of the invention may be designed with electrode pads arranged in an array format such as the array depicted in FIG. 2. Each pad may be connected to an instrument capable of applying an electrical bias. This design may permit independent control of the surface potential on each pad. A computer-based program may be used to control the bias applied to each pad based on chemical or other input.

A critical design parameter for arrays of the invention will be the inter-pad separation. The minimum separation will be that which is necessary for neighbor-independent control. As a guide, pads should be separated by a distance that is greater than or equal to the length of the nucleic acid immobilized on the pad. For example, in the case of a typical cDNA strand, this distance is approximately 1 μm.

### Example 2

A phage promoter operably linked to a gene may be used to initiate transcription. For instance, a phage promoter linked to a firefly luciferase gene may be used as a sensitive indicator of gene expression (Bronstein I et al., 1994, Anal Biochem. 219(2):169-181). The promoter-gene construct may be chemically immobilized on a conductor (e.g. gold, silver, copper, platinum, or indium tin oxide) surface. The conductor surface may be functionalized with thiol, amine, aldehyde, or avidin (a protein) groups, to which DNA chains bearing an appropriate second chemical moiety (amine, thiol, or biotin) can be cross linked directly or via a bifunctional linker molecule using standard protocols. For example, DNA amine groups can directly react with aldehyde groups on the surface or with thiol or amine surface groups using commercially available bifunctional linker molecules. Incorporation of a desired functional group into DNA can be readily achieved by amplifying the DNA in a polymerase chain reaction (PCR) using primers that bear the chemical group of interest.

DNA can be further adsorbed or repelled from the surface by controlling the electrical potential of the surface. The extent of adsorption of the DNA to the surface may be used to control its transcription. Commercial in vitro transcription as well as coupled transcription/translation systems may be employed. A potentiostat may be used to control surface potential of the conductor and, therefore, its surface charge. Transcription may be directly quantified by assaying for the messenger RNA (mRNA) product. If a luciferase gene construct is used, transcription may also be quantified by measuring the luminescence produced when luciferin, a luciferase substrate, is incubated with the mRNA translation product, i.e., luciferase produced by translating the mRNA.

### Example 3

A single-stranded DNA primer may be immobilized on a conductor surface (e.g. gold, silver, copper, platinum, or indium tin oxide), chemically or physically as in Example 2. The immobilized primer may be hybridized with longer single-stranded DNA target in solution. The primer-target complex may then be exposed to a buffer containing DNA polymerase, triphosphate nucleotides, and other reagents necessary for DNA synthesis. The binding of DNA polymerase to the primed region, and therefore the extension of the immobilized primer (by one or more nucleotides) may be controlled by varying the electrical potential applied to the conductor surface to which the primer is bound.

1. A method of controlling the contact between a surface-immobilized nucleic acid and a conductor surface comprising:
applying an electrical potential to the conductor surface so as to form surface charge thereon, wherein contact between the nucleic acid and the conductor surface is affected by the surface charge and wherein the nucleic acid remains surface-immobilized.

2. The method of claim 1, wherein a more negative surface charge decreases adsorption of the nucleic acid to the conductor surface.

3. The method of claim 1, wherein a more positive surface charge increases adsorption of the nucleic acid to the conductor surface.

4. The method of claim 1 wherein the surface-immobilized nucleic acid is covalently bound to the conductor surface.

5. The method of claim 4 wherein the surface-immobilized nucleic acid is covalently bound to the conductor surface through a thiol or amine linkage.

6. The method of claim 1 wherein the surface-immobilized nucleic acid is non-covalently bound to the conductor surface.

7. The method of claim 6 wherein the conductor surface-immobilized nucleic acid is non-covalently bound to the surface through a biotin-avidin linkage.

8. The method of claim 1 further comprising bringing the surface-immobilized nucleic acid in contact with a solvent or solution.

9. The method of claim 8 wherein the solution comprises a component selected from the group consisting of water, a buffer, a nucleic acid, a protein, an enzyme, a carbohydrate, a lipid, a salt, a salt ion, an organic chemical, a drug, a detergent, or combinations thereof.

10. The method of claim 1 further comprising bringing the surface-immobilized nucleic acid in contact with whole cell extract.

11. A method of controlling the binding of a surface-immobilized nucleic acid immobilized in contact with a conductor surface to a biomolecule comprising:
applying an electrical potential to the conductor surface so as to form surface charge thereon, wherein contact between the nucleic acid and the conductor surface is affected, and wherein the binding of the nucleic acid to the biomolecule is controlled by the contact of the nucleic acid with the conductor surface.

12. The method of claim 11, wherein a more negative surface charge decreases the adsorption of the nucleic acid to the conductor surface.

13. The method of claim 11, wherein a more positive surface charge increases the adsorption of the nucleic acid to the conductor surface.

14. The method of claim 11 wherein the biomolecule is selected from the group consisting of nucleic acids, oligonucleotides, polynucleotides, carbohydrates, lipids, amino acids, peptides, polypeptides, enzymes, drugs, and detergents.

15. Apparatus for controlling the interaction of a nucleic acid and a biomolecule comprising:
a conductor surface; and
a voltage source coupled to the conductor surface for controllably applying an electrical potential to the conductor surface so as to form surface charge thereon, wherein the nucleic acid in contact with the surface is immobilized thereon, the conformation of the nucleic acid to the conductor surface being affected by the surface charge on the conductor surface, and wherein the interaction between the immobilized nucleic acid and the biomolecule is controlled by the electrical potential applied to the conductor surface.

16. The apparatus of claim 15 wherein applying an electrical potential to the conductor surface that forms a more negative charge thereon decreases the adsorption of the nucleic acid to the conductor surface.

17. The apparatus of claim 15, wherein applying an electrical potential to the conductor surface that forms a more positive surface charge thereon increases the adsorption of the nucleic acid to the conductor surface.

18. The apparatus of claim 15, wherein the biomolecule is selected from the group consisting of nucleic acids, oligonucleotides, polynucleotides, carbohydrates, lipids, amino acids, peptides, polypeptides, enzymes, drugs, and detergents.
